(19) United States
(12) Patent Application Publication
(10) Pub. No.: US 2023/0148232 A1
(43) Pub. Date: May 11, 2023

(54) FUNCTIONAL MATERIAL AND METHOD FOR PRODUCING SAME

(71) Applicant: HONDA MOTOR CO., LTD., Tokyo (JP)

(72) Inventors: Mayu KURATA, Tochigi (JP); Takehiro MUGISHIMA, Tochigi (JP)

(21) Appl. No.: 17/913,165

(22) PCT Filed: Mar. 24, 2021
(86) PCT No.: PCT/JP2021/012315

§ 371 Date: Sep. 21, 2022

(30) Foreign Application Priority Data
Mar. 25, 2020 (JP) 2020-053888

(51) Int. Cl. A01N 59/06; A61L 2/238; A01P 1/00; A01N 25/34; C23C 22/68

(52) U.S. Cl. CPC A01N 59/06; A61L 2/238; A01P 1/00; A01N 25/34; C23C 22/68

(57) ABSTRACT

The purpose of the present invention is to provide: a functional material which is prepared by using aluminum as a base material and has a bactericidal effect; and a method for producing the functional material. The functional material comprises an aluminum base material and a functional film which is a hydrated aluminum oxide film formed on the surface of the aluminum base material. The functional film has nano-order depressions and protrusions formed on the surface thereof and, as a result, the functional film can have a bactericidal effect to kill bacteria. The functional material is produced by subjecting an aluminum base material to a boiling treatment in boiled water for a predetermined treatment time to form, on the surface of the aluminum base material, a hydrated aluminum oxide film having nano-order depressions and protrusions formed on the surface thereof.

### TECHNICAL FIELD

The present invention relates to a functional material and a method for producing the same. More specifically, the present invention relates to a functional material having a bactericidal effect to kill microorganisms (e.g., bacteria) and a method for producing the same.

### BACKGROUND ART

It is known that fine structures of nano-order recesses and protrusions, such as wings of dragonflies or cicadas and black silicon, have a bactericidal effect to kill bacteria. In recent years, functional materials having a bactericidal effect have been actively developed based on such a finding.

For example, Patent Document 1 discloses an invention relating to a synthetic polymer film having a bactericidal effect. A plurality of acicular nanopillars is formed on the surface of the synthetic polymer film disclosed in Patent Document 1, and the width of these nanopillars is in a range of 20 [nm] to 500 [nm].

Patent Document 1: Japanese Patent No. 6411962

### DISCLOSURE OF THE INVENTION

### Problems to be Solved by the Invention

As described above, the functional material disclosed in Patent Document 1 is assumed to be prepared by using a resin material as a base material, and imparting a bactericidal effect to the surface of a metal material, such as aluminum or zinc, has not been sufficiently studied.

An object of the present invention is to provide a functional material which is prepared by using aluminum as a base material and has a bactericidal effect; and a method for producing the same.

### Means for Solving the Problems

(1) A functional material according to the present invention (e.g., a functional material 1 described later) includes an aluminum base material (e.g., an aluminum base material 2 described later) and a hydrated aluminum oxide film formed on a surface of the aluminum base material (e.g., a functional film 3 described later). The hydrated aluminum oxide film has nano-order recesses and protrusions formed on the surface thereof, and has a bactericidal effect.

(2) In this case, an interval between protrusions formed on the hydrated aluminum oxide film is preferably in a range of 0.10 [μm] to 0.25 [μm].

(3) In this case, the interval between protrusions is preferably in a range of 0.17 [μm] to 0.21 [μm].

(4) In this case, an area of a recess formed on the surface of the hydrated aluminum oxide film is preferably in a range of 0.0010 [μm²] to 0.0600 [μm²].

(5) In this case, the area of a recess is preferably in a range of 0.0020 [μm²] to 0.0100 [μm²].

(6) A method for producing the functional material having a bactericidal effect according to the present invention includes subjecting an aluminum base material to a boiling treatment in boiled water to form a hydrated aluminum oxide film having nano-order recesses and protrusions formed on a surface of the aluminum base material.

### Effects of the Invention

(1) The functional material according to the present invention includes an aluminum base material and a hydrated aluminum oxide film formed on the surface of this aluminum base material, and nano-order recesses and protrusions are formed on the hydrated aluminum oxide film. The present invention can impart a stronger bactericidal effect than an aluminum base material having no nano-order recesses and protrusions as described above.

(2) The functional material according to the present invention can impart a stronger bactericidal effect by having an interval between protrusions formed on the hydrated aluminum oxide film in a range of 0.01 [μm] to 0.25 [μm].

(3) The functional material according to the present invention can impart an antifungal effect in addition to a stronger bactericidal effect by having the interval between protrusions formed on the hydrated aluminum oxide film in a range of 0.17 [μm] to 0.21 [μm].

(4) The functional material according to the present invention can impart a stronger bactericidal effect by having an area of a recess formed on the hydrated aluminum oxide film in a range of 0.001 [μm²] to 0.06 [μm²].

(5) The functional material according to the present invention can impart an antifungal effect in addition to a stronger bactericidal effect by having the area of a recess formed on the hydrated aluminum oxide film in a range of 0.003 [μm²] to 0.01 [μm²].

(6) In the method for producing a functional material according to the present invention, the aluminum base material is subjected to a boiling treatment in boiled water to form a hydrated aluminum oxide film having nano-order recesses and protrusions formed on the surface of the aluminum base material. This enables the formation of the hydrated aluminum oxide film having a bactericidal effect on the surface of the aluminum base material in a simple procedure.

### BRIEF DESCRIPTION OF THE DRAWINGS

FIG. 1 is a perspective view illustrating a configuration of a functional material according to one embodiment of the present invention;

FIG. 2 is a diagram illustrating steps of producing a functional material;

FIG. 3 shows SEM images of surfaces of functional films of Comparative Example 1 and Examples 1 to 5 as observed by a scanning electron microscope;

FIG. 4 is a view for describing a procedure of calculating an interval between adjacent protrusions;

FIG. 5 shows results of sterilization tests for Comparative Example 1 and Examples 1 to 5; and

FIG. 6 shows results of antifungal tests for Comparative Example 1 and Examples 1 to 5.

### PREFERRED MODE FOR CARRYING OUT THE INVENTION

Hereinafter, a functional material and a method for producing the same according to one embodiment of the present invention will be described with reference to the drawings.

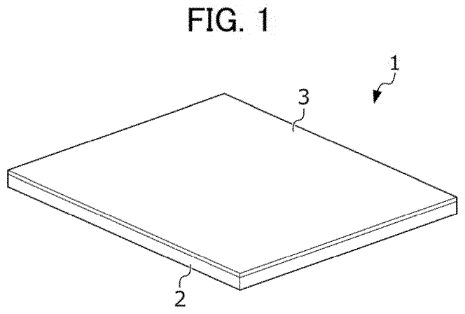

FIG. 1 is a perspective view illustrating a configuration of a functional material 1 according to the present embodiment. The functional material 1 includes a flat aluminum base material 2 and a functional film 3 formed on the surface of the base material 2.

The aluminum base material 2 is a flat plate material composed of aluminum or an aluminum alloy containing copper, manganese, silicon, magnesium, zinc, nickel, and the like with aluminum as a main component. Note that the aluminum base material 2 is described below as the flat plate material composed of aluminum or the aluminum alloy, but the present invention is not limited thereto. The shape of the aluminum base material 2 is not limited to a flat plate but may be any shape that suits its application.

The functional film 3 is a hydrated aluminum oxide film, and numerous nano-order (specifically, in a range of 1 [nm] to 1,000 [nm]) recesses and protrusions in fine and irregular shapes are formed on the surface thereof. The numerous protrusions formed on the functional film 3 are blade-shaped, and their orientations in planar view are irregular. On the functional film 3, numerous recesses are formed as a concave space defined by a plurality of these protrusions. The interval between the adjacent protrusions in planar view, in other words, the length of one side of the depression recess in planar view is of nano-order. As will be described in detail later, the functional film 3 has a bactericidal effect to kill microorganisms (e.g., bacteria).

Note that in the present invention, the function of killing bacteria is referred to as a bactericidal effect, and the function of preventing the growth of mold is referred to as an antifungal effect. Hereafter, the functional material having a stronger bactericidal effect when compared to a material of Comparative Example 1 described later is also referred to as a bactericidal material. The functional material having a stronger antifungal effect when compared to the material of Comparative Example 1 is also referred to as an antifungal material. In addition, the functional material having stronger bactericidal and antifungal effects when compared to the material of Comparative Example 1 is also referred to as a bactericidal/antifungal material.

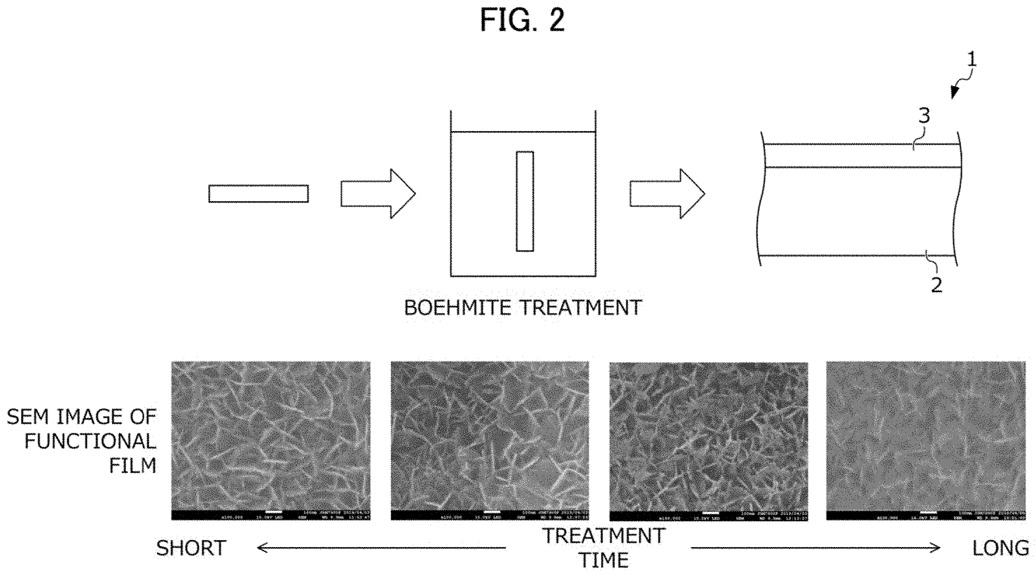

FIG. 2 is a diagram illustrating steps of producing a functional material 1. As illustrated in FIG. 2, the functional material 1 is produced by subjecting an aluminum base material 2 to a boehmite treatment. More specifically, steps of producing the functional material 1 include: preparing an aluminum base material 2 as a raw material; washing, degreasing, and rinsing the prepared aluminum base material 2; and subjecting the washed aluminum base material 2 to a boiling treatment in boiled pure water for a predetermined treatment time to form a functional film 3 as a hydrated aluminum oxide film having nano-order recesses and protrusions (see cross-sectional diagram illustrated in the upper row of FIG. 2) formed on the surface of the aluminum base material 2. As illustrated in the lower row of FIG. 2, the shape and size of the recesses and protrusions formed on the functional film 3 can be varied by changing the treatment time of the boiling treatment. Note that the functional material 3 formed on the surface of the aluminum base material 2 by the above-mentioned boehmite treatment could not be peeled off at least by hand.

Next, the contents of sterilization and antifungal tests conducted to verify the bactericidal and antifungal effects of the functional material 1 according to the present embodiment will be described, as well as Comparative Example 1 and Examples 1 to 5 used in these tests.

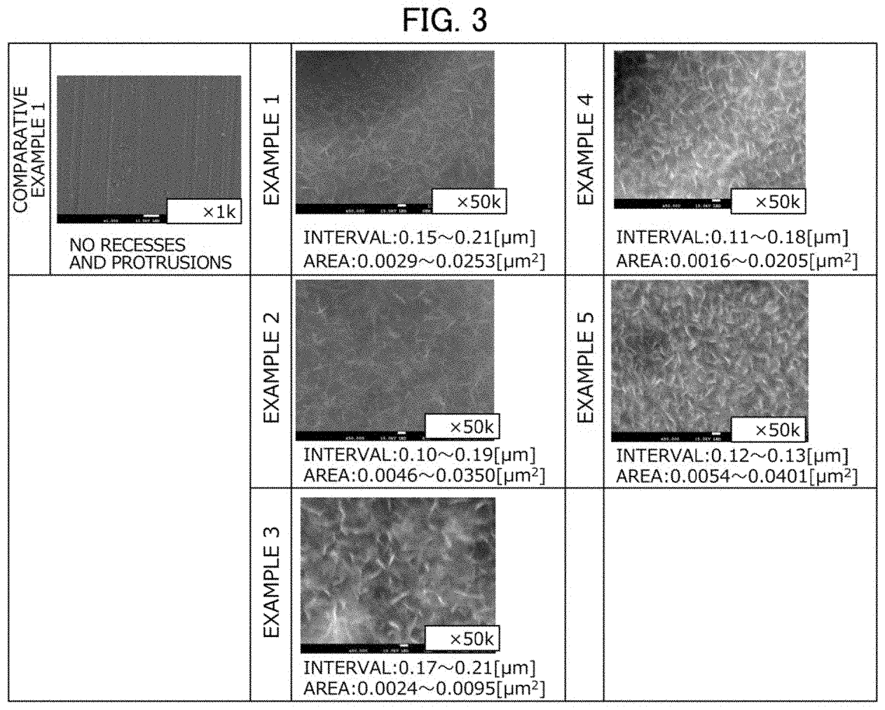

FIG. 3 shows SEM images of the surfaces of the functional films of Comparative Example 1 and Examples 1 to 5 taken under magnification by a scanning electron microscopy. FIG. 3 also shows the minimum and maximum values of the interval [μm] between adjacent portions in a plurality of protrusions formed on the surfaces of functional films along with the magnification of each SEM image.

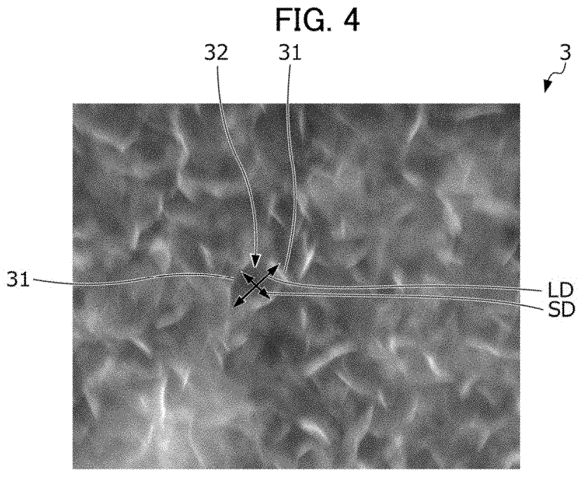

FIG. 4 is a view for describing a procedure of calculating the interval between adjacent protrusions. As shown in FIG. 4, numerous blade-shaped protrusions 31 (portions seen brightly in FIG. 4) with irregular orientations are formed on the surface of the functional film 3. Therefore, on the surface of the functional film 3, numerous recesses 32 (portions seen dark in FIG. 4) are formed as a concave space defined by a plurality of these protrusions 31.

In the present invention, the length of one side of the recess 32 in planar view is defined as an interval between the adjacent protrusions 31 in planar view. More specifically, the shape of each recess 32 formed on the surface of the functional film 3 in planar view is regarded as a shape capable of defining a longitudinal direction LD and a shorter direction SD orthogonal thereto (e.g., a rectangular shape or an elliptical shape), the longitudinal direction LD and the shorter direction SD orthogonal thereto are defined for the recesses 32, and the lengths of the recesses 32 along the longitudinal direction LD and the shorter direction SD are defined as the interval between the adjacent protrusions 31. The length of each recess 32 along the longitudinal direction LD and the shorter direction SD is calculated based on the above definitions, and the minimum length along the shorter direction SD is the minimum interval between the protrusions 31 while the maximum length along the longitudinal direction LD is the maximum interval between the protrusions 31.

### Comparative Example 1

In the above tests, an aluminum base material that has not undergone the boehmite treatment as described above was designated as Comparative Example 1. A3000 series (aluminum-manganese alloy) was used as the aluminum base material. As shown in FIG. 3, since a functional material of Comparative Example 1 has not undergone the boehmite treatment, no hydrated aluminum oxide film is formed on the surface thereof, and it is also substantially flat and has no recesses and protrusions.

### Example 1

In the above tests, the same aluminum base material (i.e., A3000 series) as used in Comparative Example 1 was subjected to the boehmite treatment for a treatment time of 10 minutes and designated as Example 1. As shown in FIG. 3, an interval between protrusions formed on a functional film of Example 1 was in a range of 0.15 [μm] to 0.21 [μm]. An area of a recess was in a range of 0.0029 [μm²] to 0.0253 [μm)].

### Example 2

In the above tests, the same aluminum base material (i.e., A3000 series) as used in Comparative Example 1 was subjected to the boehmite treatment for a treatment time of 15 minutes and designated as Example 2. As shown in FIG. 3, an interval between protrusions formed on a functional film of Example 2 was in a range of 0.10 [μm] to 0.19 [μm]. An area of a recess was in a range of 0.0046 [μm²] to 0.0350 [μm²].

### Example 3

In the above tests, the same aluminum base material (i.e., A3000 series) as used in Comparative Example 1 was subjected to the boehmite treatment for a treatment time of 30 minutes and designated as Example 3. As shown in FIG. 3, an interval between protrusions formed on a functional film of Example 3 was in a range of 0.17 [μm] to 0.21 [μm]. An area of a recess was in a range of 0.0024 [μm²] to 0.0095 [μm²].

### Example 4

In the above tests, the same aluminum base material (i.e., A3000 series) as used in Comparative Example 1 was subjected to the boehmite treatment for a treatment time of 45 minutes and designated as Example 4. As shown in FIG. 3, an interval between protrusions formed on a functional film of Example 4 was in a range of 0.11 [μm] to 0.18 [μm]. An area of a depression was in a range of 0.0016 [μm²] to 0.0205 [μm²].

### Example 5

In the above tests, the same aluminum base material (i.e., A3000 series) as used in Comparative Example 1 was subjected to the boehmite treatment for a treatment time of 60 minutes and designated as Example 5. As shown in FIG. 3, an interval between protrusions formed on a functional film of Example 5 was in a range of 0.12 [μm] to 0.13 [μm]. An area of a depression was in a range of 0.0054 [μm²] to 0.0401 [μm²].

### <Sterilization Test>

In sterilization tests, the function of killing Escherichia coli, a gram-negative bacterium, was verified for Comparative Example 1 and Examples 1 to 5. More specifically, a solution of Escherichia coli (10⁷ cells/ml) was added dropwise onto the surface of the functional film and allowed to stand still for 1 hour to settle the bacterial cells. After 30 minutes, the solution of Escherichia coli added dropwise onto the functional film was recovered, and the recovered solution of Escherichia coli was evaluated by a colony count method to calculate a sterilization rate [%] after a lapse of 1.5 hours from the dropwise addition. The colony count method is a method for calculating the number of viable bacteria in the recovered solution by measuring the number of colonies generated after the viable bacteria in the solution have been cultured for a predetermined time.

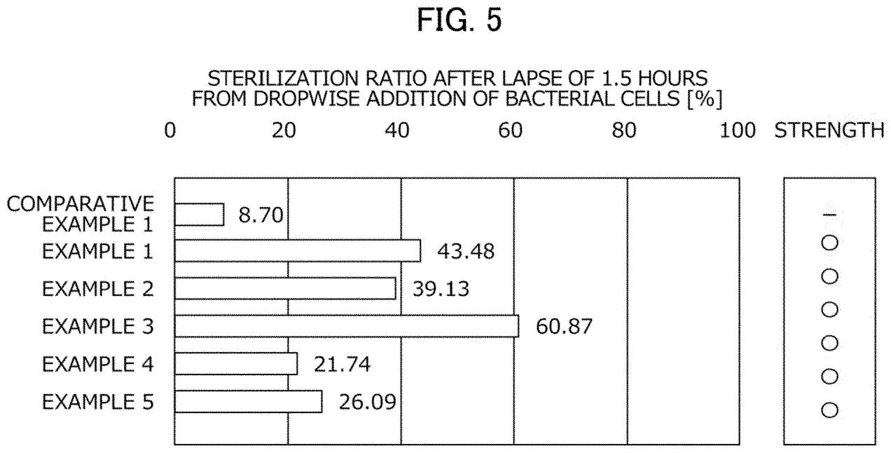

FIG. 5 shows results of sterilization tests for Comparative Example 1 and Examples 1 to 5. In FIG. 5, the left side shows the results of the above sterilization tests while the right side shows whether the functional films peel off after the sterilization tests. As shown in FIG. 5, the sterilization rate [%] at 1.5 hours after the dropwise addition of the bacterial cells onto the surface of the functional film was 8.70 [%] in Comparative Example 1, 43.48 [%] in Example 1, 39.13 [%] in Example 2, 60.87 [%] in Example 3, 21.74 [%] in Example 4, and 26.09 [%] in Example 5. Examples 1 to 5 have higher sterilization rates than Comparative Examples 1 when compared in this manner. Therefore, it was verified that the functional materials of Examples 1 to 5, in which the interval between the protrusions is in the range of 0.10 [μm] to 0.25 [μm] and the area of the recess is in the range of 0.0010 [μm²] to 0.0600 [μm²], can be used as bactericidal materials. In particular, it was verified that the functional material of Example 3, in which the interval between the protrusions is in the range of 0.17 [μm] to 0.21 [μm] and the area of the recess is in the range of 0.0020 [μm²] to 0.0100 [μm²], has a particularly stronger bactericidal effect than the other functional materials of Examples 1, 2, 4, and 5.

As shown in FIG. 5, none of the functional films of Examples 1 to 5 were peeled off after the sterilization tests. Therefore, it was also verified that the functional films, the hydrated aluminum oxide films, in the functional materials of Examples 1 to 5 each have sufficient strength.

### <Antifungal Test>

In antifungal tests, the function of preventing the growth of Aspergillus niger was verified for Comparative Example 1 and Examples 1 to 5. More specifically, a spore suspension of Aspergillus niger was prepared so as to have a predetermined concentration, and this spore suspension was cultured on the surfaces of the functional films to determine at the predetermined intervals whether mycelial growth was visually or microscopically observed.

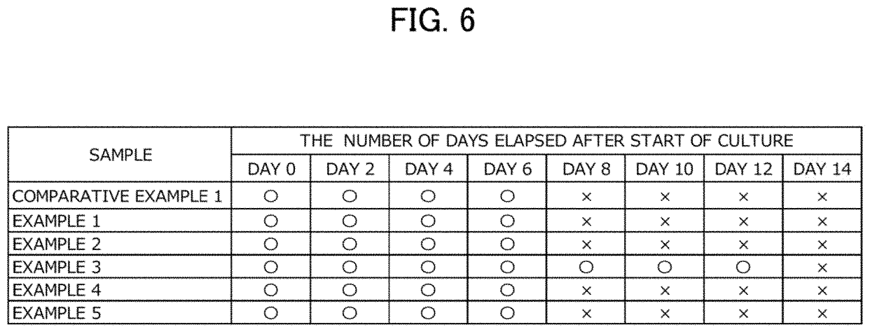

FIG. 6 shows results of antifungal tests for Comparative Example 1 and Examples 1 to 5. In FIG. 6, “o (circle symbol)” indicates that no mycelial growth was observed, while “x (cross symbol)” indicates that mycelial growth was observed.

As shown in FIG. 6, mycelial growth was seen on day 8 in Comparative Example 1, mycelial growth was seen on day 8 in Example 1, 2, 4, and 5, and mycelial growth was seen on day 14 in Example 3. The number of days taken for mycelial growth to be seen is almost the same in Examples 1, 2, 4, and 5 as in Comparative Example 1, and longer in Example 3 than in Comparative Example 1 when compared in this manner. Therefore, it was verified that the functional materials of Examples 3, in which the interval between the protrusions is in the range of 0.17 [μm] to 0.21 [μm] and the area of the recess is in the range of 0.0020 [μm²] to 0.0100 [μm²], can be used as an antifungal material. It was also verified that the functional material of Example 3 has a slightly stronger antifungal effect than the other functional materials of Examples 1, 2, 4, and 5.

### EXPLANATION OF REFERENCE NUMERALS

1 FUNCTIONAL MATERIAL
2 ALUMINUM BASE MATERIAL
3 FUNCTIONAL FILM (HYDRATED ALUMINUM OXIDE FILM)

1. A functional material comprising:
an aluminum base material and
a hydrated aluminum oxide film formed on a surface of the aluminum base material, wherein
nano-order recesses and protrusions are formed on the hydrated aluminum oxide film, and
the functional material has a bactericidal effect.

2. The functional material according to claim 1, having an interval between protrusions formed on the hydrated aluminum oxide film in a range of 0.10 [μm] to 0.25 [μm].

3. The functional material according to claim 2, having the Interval between protrusions in a range of 0.17 [μm] to 0.21 [μm].

4. The functional material according to claim 1, having an area of a recess formed on the hydrated aluminum oxide film in a range of 0.0010 [μm²] to 0.0600 [μm²].

5. The functional material according to claim 4, having the area of a recess in a range of 0.0020 [μm²] to 0.0100 [μm²].

6. A method for producing a functional material having a bactericidal effect, comprising subjecting an aluminum base material to a boiling treatment in boiled water to form a hydrated aluminum oxide film having nano-order recesses and protrusions on a surface of the aluminum base material.

7. The functional material according to claim 2, having an area of a recess formed on the hydrated aluminum oxide film in a range of 0.0010 [μm²] to 0.0600 [μm²].

8. The functional material according to claim 7, having the area of a recess in a range of 0.0020 [μm²] to 0.0100 [μm²].
